(19) United States
(12) Patent Application Publication
(10) Pub. No.: US 2009/0218229 A1
(43) Pub. Date: Sep. 3, 2009

(54) COATING PROCESS AND APPARATUS WITH IMPROVED RESISTANCE TO BACTERIA

(75) Inventors: Timothy J. SHREWSBURG, Ansonia, OH (US); Richard E. DeSHEPPER, Southgate, MI (US)

Correspondence Address: Harness, Dickey and Pierce, P.L.C., 5445 Corporate Drive, Troy, MI, 48098, US

(73) Assignee: BASF CORPORATION, Wyandotte, MI (US)

(21) Appl. No.: 12/463,868

(22) Filed: May 11, 2009

Related U.S. Application Data
(62) Division of application No. 10/795,735, filed on Jun. 17, 2004.

(51) Int. Cl. C25D 5/00; C25D 3/02; A61L 2/04

(52) U.S. Cl. 205/80; 422/1

(57) ABSTRACT

A coating process using an aqueous coating or aqueous rinse includes a step in which at least a part of the aqueous coating, aqueous rinse, and/or an aqueous component added to the aqueous coating or aqueous rinse is pasteurized. The pasteurization may be carried out continuously in a circulation loop of a tank containing the material or in a line carrying the material to a sprayhead.

### FIELD OF THE INVENTION

The invention relates methods for preventing or reducing bacteria growth in coating processes using aqueous materials, such as aqueous wash processes and aqueous coating composition baths. In certain particular applications, the invention concerns preventing or reducing bacteria growth in aqueous rinse water following phosphate coating or electrodeposition coating processes and in electrodeposition coating tanks.

### BACKGROUND OF THE INVENTION

Industrial coating of metal articles that will be used in corrosive environments may include application of one or more inorganic and organic treatments and coatings. Steel automotive vehicle bodies and parts, for instance, have an aqueous phosphate coating material applied, are rinsed with rinse water after phosphating, then have an aqueous electrodeposition (or electrocoat) coating applied, followed by multiple aqueous rinses before the electrodeposited coating is cured in an oven.

Bacteria growth in aqueous materials used for industrial coating processes like coating of automotive bodies and parts interferes with the quality and effectiveness of the aqueous materials. Bacteria growth in rinse water reduces rinsing efficiency. Bacteria growth in electrocoat coating composition tanks can cause plating problems and film defects, or even reduce the corrosion protection of the electrodeposited coating.

Biocides and other agents, such as hydrogen peroxide, have been added to pretreatment rinse water, electrocoat coating tanks, and electrocoat rinse water to destroy bacteria. These materials reduce the amount of bacteria, but in time, they lose activity. The added biocide materials are also removed over time as the rinse or electrocoat coating is used. The bacteria level must be continually monitored, and new biocide material must be added periodically to respond to increased bacteria levels. This complicates and adds expense to the coating process and requires additional material handling.

It would thus be desirable to introduce an improved way of controlling bacteria growth in the aqueous materials used in coatings processes.

### SUMMARY OF THE INVENTION

The present invention provides a method of coating a substrate in which one or more of an aqueous coating composition, an aqueous rinse, or an aqueous component added to an aqueous coating composition or aqueous rinse is pasteurized. If an aqueous coating composition is pasteurized, it is then used to apply a coating layer onto a substrate. If an aqueous component for a coating composition is pasteurized, it is then added to a coating composition that is used to apply a coating layer onto a substrate. If an aqueous rinse is pasteurized, it is then used to rinse a substrate or a coating layer that has been applied onto a substrate. If an aqueous component for an aqueous rinse is pasteurized, it is then added to an aqueous rinse used to rinse a substrate or a coating layer that has been applied onto a substrate. The aqueous coating composition, rinse, or component is pasteurized by heating it in order to destroy bacteria. Preferably, substantially all of the bacteria is destroyed and, more preferably, all of the bacteria is destroyed by the pasteurization.

The invention further provides an apparatus that includes a container of fluid comprising water an aqueous coating material, wherein the container has at least first and second apertures, the first aperture being below the fluid level in the container and a pipeline extending from the first aperture to the second aperture, said pipeline including a fluid pump that moves a portion of the fluid from the first aperture to the second aperture, a heater that heats the fluid in the pipeline to a temperature sufficient to pasteurize the fluid, and, optionally, a chiller for cooling the pasteurized fluid to a desired temperature.

The invention further provides a coating line including a tank of an aqueous material, wherein the tank has a circulation loop containing a heater that heats the aqueous material passing through the circulation loop to a temperature sufficient to pasteurize the fluid, and, optionally, a chiller for cooling the pasteurized fluid to a desired temperature.

The invention further provides an apparatus for spray application of an aqueous coating composition or for spray of an aqueous rinse material, wherein the apparatus includes a line for conveying the aqueous coating composition or aqueous rinse material for spraying, and in which the line includes a heater for pasteurizing the aqueous coating composition or aqueous rinse material and, optionally, a chiller for cooling the pasteurized aqueous coating composition or aqueous rinse material.

The invention further provides a method of pasteurizing an aqueous coating material or rinse in which the aqueous coating material or rinse is heated to at least substantially destroy bacteria in the aqueous coating material or rinse, then the pasteurized coating material or rinse is added to an aqueous coating tank or aqueous rinse in a coating line.

The invention further provides a method of preventing bacteria contamination in a coating line by pasteurizing phosphate rinse water so that bacteria is not carried into an electrocoat bath.

“A” and “an” as used herein indicate “at least one” of the item is present; a plurality of such items may be present, when possible. “About” when applied to values indicates that the calculation or the measurement allows some slight imprecision in the value (with some approach to exactness in the value; approximately or reasonably close to the value; nearly). If, for some reason, the imprecision provided by “about” is not otherwise understood in the art with this ordinary meaning, then “about” as used herein indicates a possible variation of up to 5% in the value.

### BRIEF DESCRIPTION OF THE DRAWINGS

The present invention will become more fully understood from the detailed description and the accompanying drawings, wherein:

The FIGURE is a functional block diagram of a system for pasteurizing an aqueous coating rinse or coating material.

### DETAILED DESCRIPTION OF THE PREFERRED EMBODIMENTS

The following description of the preferred embodiment(s) is merely exemplary in nature and is in no way intended to limit the invention, its application, or its uses.

Coating of a vehicle includes steps for preparing the vehicle body metal by cleaning and rinsing, applying an aqueous phosphate treatment to the metal, rinsing, electrodeposition priming, rinsing, baking, and application of further coating layers, one or more of which may be aqueous. Dip tanks, such as for applying the phosphate treatment or the electrodeposition primer, have recirculation loops. A heater for pasteurizing the aqueous material in the tank can be put into a recirculation loop. The heater preferably heats the material quickly to the pasteurization temperature, and preferably is followed in the line by a chiller that quickly cools the pasteurized material to a desired temperature. Spray equipment for spray applying an aqueous coating composition or for rinsing with an aqueous rinse will include a fluid line for conveying the coating composition or rinse from a reservoir or storage container to the spray head. A heater for pasteurizing the aqueous coating composition or rinse can be inserted in the fluid line. Again, the heater preferably heats the coating or rinse quickly to the pasteurization temperature, and preferably is followed in the line by a chiller that quickly cools the pasteurized coating or rinse to a desired temperature.

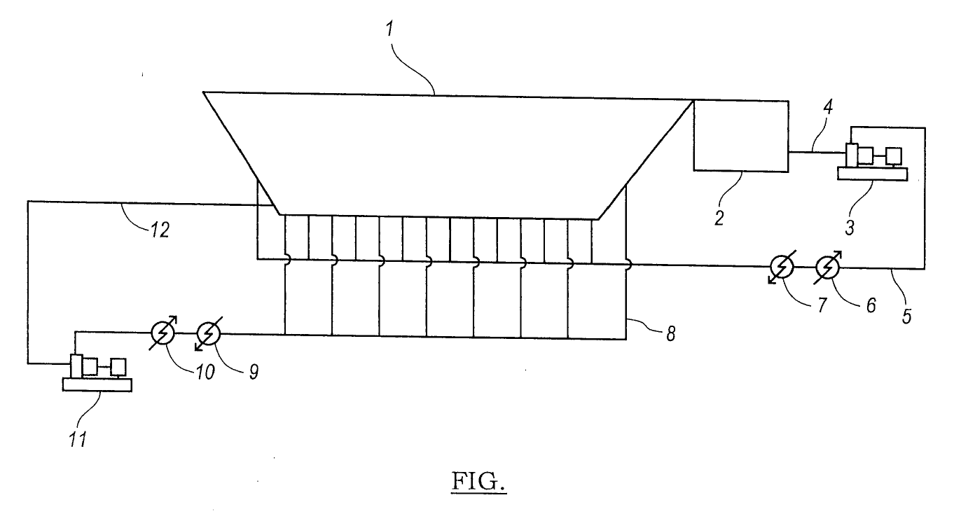

Referring to FIG. 1, in one embodiment of the invention a tank 1 containing an aqueous coating rinse or coating material has an overflow weir 2. Pump 3 draws aqueous overflow material from near the bottom of weir through pipe 4 connecting the weir to the pump and forces the overflow material through pipeline 5, which returns the overflow material to tank 1 through an opening in the side of tank 1 near the bottom. Pipeline 5 includes a heat exchanger 6 followed by a chiller 7. The heat exchanger 6 heats the overflow material to a temperature sufficient for pasteurizing the overflow material. The chiller 7 cools the heated overflow material to a desired temperature, such as to a temperature that does not tend to increase the temperature of the aqueous coating rinse or coating material in tank 1.

Tank 1 is also shown with filter pipeline 12 through which aqueous coating rinse or coating material from tank 1 is drawn by pump 11. The coating rinse or coating material passes through heat exchanger 10 and then chiller 9. The heat exchanger 10 heats the coating rinse or coating material to a temperature sufficient for pasteurizing the coating rinse or coating material. The chiller 9 then cools the heated coating rinse or coating material to a desired temperature, such as to a temperature that does not tend to increase the temperature of the aqueous coating rinse or coating material in tank 1. The coating rinse or coating material is then pumped through filters [not shown] in multiple lines 8 that return the coating rinse or coating material to tank 1.

In general, an aqueous rinse or coating material may be pasteurized by heating as quickly as possible to a pasteurization temperature of about 125° F. or higher. Heat may be an issue with pigmented coating because of destabilization of pigment dispersion; the pasteurization temperature is preferably as low as possible for pigmented materials. The pasteurization temperature should not be so high that the material or equipment is adversely affected (e.g., destabilized material that has poor properties or causes settling that blocks the pipelines), or is heated to a temperature so high chiller cannot adequately cool the material afterward. In general, the material may be heated to at least about 125° F., more preferably at least about 130° F., and may be heated up to about 145° F., generally as quickly as possible, as in about 2 to 3 seconds to about 2 to 3 minutes.

Heat exchangers are well known in manufacturing and coating processes. Heat exchangers are available in various designs, such as a jacketed pipe, a plate-and-frame heat exchanger, HTST continuous plate pasteurizer, shell and tube design heat exchanger, and so on. The heat may be provided by hot water, steam, heated brine, glycol, heat transfer oils or other heat transfer fluids, electric induction, or suitable heating methods.

A chiller is preferably located in the fluid line or circulation pipeline shortly after the heat exchanger so that the coating rinse or coating material is not at the pasteurization temperature so long that it is adversely affected. If the pasteurized material is not thermally sensitive, a chiller may not be necessary. The chiller may be of the same or different design as the heat exchanger, with the cooling provided by cold water, glycol, brine, heat transfer oils or other heat transfer fluids, or via other suitable cooling mean. Preferably, a coating material that is pasteurized is cooled substantially immediately after leaving the heat exchanger. Rinse material may not need to be cooled at all, unless it contains material that heat may tend to destabilize so that it deposits in the pipeline or it becomes so heated that it removes or otherwise adversely affects the applied coating layer that it rinses.

In a continuous process of the invention, the aqueous material is treated by continuous removal of a portion of the aqueous material from a tank or other container, pasteurization of the portion removed, and return of the pasteurized material to the tank or container. A pasteurization device, such as a combination of a heat exchanger followed by a chiller, is installed in the continuous circulation loop. The rate at which material is pasteurized in the loop may depend on the circulation rate and turnover rate of the tank, as the pasteurization device may be inserted into circulation equipment for the tank.

In a continuous pasteurization process, any bacteria may be continuously destroyed so that bacteria never increase to deleterious levels. Performance and stability of the aqueous material may be enhanced by preventing or reducing bacteria outbreaks.

Aqueous material in storage vessels, such as water storage vessels, may become infested with bacteria because the material is stagnant allowing growth of bacteria colonies present when the material is put into storage. The storage tanks may also become infected through the inerting process for the tank. Storage tanks are typically connected to a gas source and a relief device that ensures a safe pressure is maintained in the tank during its operation. Bulk storage tanks may have conservation vents that vent to a thermal oxidizer or to the atmosphere. Bulk storage tanks may also have vacuum breakers, again supplied by inert gas or atmosphere. When material is added or removed, air-containing bacteria may be introduced into the tank. Thus, it may be desirable to pasteurize the aqueous material before adding it to a coating or rinse by passing the aqueous material through a pasteurization device as described above.

In a preferred embodiment, an aqueous phosphate coating, a metal pretreatment rinse, or an electrocoat coating or rinse following an electrodeposition step is pasteurized. In typical assembly lines including an electrocoat coating bath, the metal substrate (e.g., a vehicle body or part) is first pretreated by a phosphate coating system. The metal substrate is cleaned, rinsed, then dipped in a phosphate coating bath or sprayed by a phosphate coating, and finally rinsed with an aqueous rinse. The treated metal substrate next enters an electrodeposition coating bath, where a coating layer is electrophoretically deposited onto the substrate. Pasteurizing the phosphate treatment rinse water prevents bacteria being carried into the electrodeposition coating bath from rinse water remaining on the metal substrate. When the coated substrate leaves the electrocoat bath, it is again rinsed with water. In a typical electrocoat process, the coated substrate is rinsed in three separate rinses. Pasteurizing an electrocoat coating rinse, or all of the rinses, prevents bacteria being carried into the electrodeposition coating bath from backflow of the rinse water.

The description of the invention is merely exemplary in nature and, thus, variations that do not depart from the gist of the invention are intended to be within the scope of the invention. Such variations are not to be regarded as a departure from the spirit and scope of the invention.

1. A method of coating a substrate, comprising a step in which at least a part of one or more aqueous materials selected from the group consisting of:
aqueous coating compositions, aqueous rinses, and aqueous components added to
aqueous coating compositions and aqueous rinses
is pasteurized.

2. A method according to claim 1, wherein at least a part of an aqueous coating composition is pasteurized and the aqueous coating composition is used to apply a coating layer onto a substrate.

3. A method according to claim 2, wherein the aqueous coating composition is an aqueous phosphate coating composition.

4. A method according to claim 2, wherein the aqueous coating composition is an electrocoat coating composition and the coating layer is applied to the substrate by electrodeposition coating.

5. A method according to claim 1, wherein an aqueous component for a coating composition is pasteurized, and further wherein the aqueous component is then added to a coating composition that is used to apply a coating layer onto a substrate.

6. A method according to claim 1, wherein at least a part of an aqueous rinse is pasteurized and further wherein the pasteurized aqueous rinse is used to rinse a substrate or a coating layer that has been applied onto a substrate.

7. A method according to claim 6, wherein the pasteurized aqueous rinse is used to rinse a coating layer that has been applied onto a substrate by electrodeposition coating.

8. A method according to claim 1, wherein at least a part of an aqueous rinse is pasteurized and further wherein the pasteurized aqueous rinse is used as a metal pretreatment rinse.

9. A method according to claim 1, wherein an aqueous component for an aqueous rinse is pasteurized and the pasteurized component for an aqueous rinse is then added to an aqueous rinse that is used to rinse a substrate or a coating layer that has been applied onto a substrate.

10. A method according to claim 1, wherein all bacteria of the at least part of one or more materials that are pasteurized are destroyed by the pasteurization.

11. A method according to claim 1, wherein the aqueous material is pasteurized by continuously removing a portion of the aqueous material from a tank or other container, pasteurizing the portion removed, and returning the pasteurized material to the tank or container.

12. A method according to claim 11, wherein the aqueous material is passed through a pipeline, said pipeline including a heat exchanger in which the aqueous material is heated to a temperature sufficient for pasteurization.

13. A method according to claim 11, wherein the heated aqueous material is cooled in a chiller in the pipeline downstream of the heat exchanger.

14. (canceled)

15. A method of preventing bacteria contamination in a coating line, comprising a step of pasteurizing phosphate rinse water so that bacteria is not carried into an electrocoat bath.

16-23. (canceled)

24. A method of preventing or removing bacterial contamination in a coating line, comprising a step of pasteurizing at least a portion of an aqueous coating composition.

25. A method of preventing or removing bacterial contamination in a coating line according to claim 24, wherein the aqueous coating composition is a phosphate coating composition that is applied in the coating line.

26. A method of preventing or removing bacterial contamination in a coating line according to claim 24, wherein the aqueous coating composition is an electrocoat coating composition contained in an electrodeposition tank in the coating line.

27. A method of preventing or removing bacterial contamination in a coating line according to claim 24, wherein the aqueous coating composition is an aqueous coating composition that is applied to a substrate by spray application in the coating line.

28. A method of preventing or removing bacterial contamination in a coating line according to claim 24, wherein the at least a portion of an aqueous coating composition is pasteurized at a temperature of about 125° F. or higher.
